(19) United States
(12) Patent Application Publication
(10) Pub. No.: US 2026/0348817 A1
(43) Pub. Date: Nov. 5, 2026

(54) PERFECTED METHOD FOR THE ANALYSIS OF A LIQUID WITH SUSPENDED BODIES

(71) Applicant: ALIFAX S.R.L., Polverara (IT)

(72) Inventor: Paolo Galiano, Padova (IT)

(73) Assignee: ALIFAX S.R.L., Polverara (IT)

(21) Appl. No.: 17/757,940

(22) PCT Filed: Dec. 22, 2020
(86) PCT No.: PCT/IT2020/050321

§ 371 Date: Jun. 23, 2022

(30) Foreign Application Priority Data
Dec. 23, 2019 (IT) 102019000025444

(51) Int. Cl. G01N 15/05; G01N 11/00; G01N 33/49

(52) U.S. Cl. CPC G01N 15/05; G01N 11/00; G01N 33/49; G01N 2011/008; G01N 2015/055

(57) ABSTRACT

A method for the analysis of a liquid with suspended bodies, such as blood which specifically analyzes the behavior of a liquid containing suspended bodies subjected to stress in order to obtain cognitive factors connected to the liquid itself and to the suspended bodies. In particular, an exemplary aspect of the present invention is to carry out the analysis of the balances reached in said liquid by the suspended bodies. The method finds application in a wide form of fluids.

### FIELD OF THE INVENTION

The present invention concerns a method for the analysis of a liquid with suspended bodies.

The purpose of the present invention is to analyze the behavior of suspended bodies present in a liquid in order to obtain cognitive factors connected to the liquid itself, to the suspended bodies and to the system of which the liquid is part.

In particular, the purpose of the present invention is to analyze the equilibria reached in a liquid, which is normally in motion according to a defined path, of the suspended bodies deformed by stresses, when the liquid is stopped.

The present invention is applied in a wide range of liquids, in particular, although not restrictively, animal blood, especially human blood.

The present invention uses quantitative capillary photometry associated with a fixed measurement window, the liquid coming directly from a fixed pipe.

Another object of the present invention is a method of analysis which allows to obtain a series of types of evaluations and measurements connected with the specific liquid, performed using a temporary stop, having a minimum duration, of a minimum quantity of the liquid in a position and in conditions such as to be the subject of such an analysis, said minimum quantity being preceded and followed by separation means that separate it from the preceding and subsequent quantities.

The present invention also concerns the modified definition of the sedimentation curve, in particular connected to human blood.

The present invention therefore allows, in the case of blood for example, to detect, together or in parallel with the evaluation of the Erythrocyte Sedimentation Rate (ESR), other factors inherent to the blood, for example one or more of the following characteristic factors: viscosity (Vs), red blood cell elasticity (Btp), blood density (Ds), hematocrit value (Hct), hemoglobin (Hb), anemia factor (AnF) and aggregation factor (AgF).

### BACKGROUND OF THE INVENTION

Various methods are known for the analysis of bodies suspended in a liquid. For example, for blood, the ESR index provides data relating to the sedimentation rate of erythrocytes. This index is obtained with a test that is normally carried out, in the case of blood, with the blood conditioned in a specific situation. The blood test can be obtained both in the case where the blood is rendered incoagulable and also with whole blood without anti-coagulant.

When we consider below the case of application to human blood, the traditional methods for testing it are the known Wintrobe and Westergreen methods, which require about an hour to provide the desired result.

The test is detected by sedimentation of the blood, rendered incoagulable, in hollow rods or test tubes of various composition, glass or plastic, which supply the measurement of the non-corpuscular part. With these known methods, after sedimentation, there is a clear part and a corpuscular part, in which the height of the measurement on a calibrated pipette of the non-corpuscular part is defined as ESR, or Erythrocyte Sedimentation Rate.

From U.S. Pat. No. 3,679,367 it is known to introduce packets of blood under pressure into a serpentine capillary pipe present in a rotating centrifugation disc, forcing the blood segments to travel along the serpentine path. As the disc continues to rotate, with this system using collimated light, the volume of packed cells of each whole blood sample is measured.

WO-A-92/09879 provides a device to rapidly perform a sedimentation rate test, comprising a support for an elongated container equipped with a lid for receiving a determined quantity of blood. The support can be rotated in the vertical plane about an axis perpendicular to its longitudinal axis, for a predetermined number of revolutions at a given speed. The container is then stopped in a position where its longitudinal axis forms a predetermined angle with the vertical for a determined period of time, then the container is rotated at a predetermined speed about the first axis and about the site where the sedimentation rate is measured.

DE-U-9216127 is also similar.

In WO-A-94/18557 an apparatus for analyzing samples is provided, comprising means for transporting the sample, a centrifuge for rotating the sample at a predetermined speed of rotation such that the centrifugal force separates the various phases or components of the sample. Also comprised are sample reading means, to read or measure predetermined characteristics of the sample, analysis means to analyze the data obtained by the sample reading means, and display means to display the data produced by the analysis means relating to the sample.

U.S. Pat. No. 4,135,819 shows a method to obtain the measured values of blood sedimentation corresponding to blood subsidence. A blood sample is inserted in a transparent measuring chamber, removing the erythrocyte aggregation present in the sample, illuminating the sample with light and measuring the quantity of light that allows the sample to pass for a determined time. The sample is inserted into a measuring chamber having upper and lower means mobile with respect to each other, configured to come into contact with the sample. The aggregation of erythrocytes present in the operation is contrasted by the movement of the upper means with respect to the lower means. The quantity of light that comes out of the sample during the aggregation process is measured photometrically after a sudden stop. With this method, a sylectogram is obtained from the photometric signals, which defines the start of the aggregation phase within a predetermined time interval of about 5 seconds after the sudden stop. Test values are defined at a predetermined time within approximately 2.5 seconds after the start of the blood aggregation. From the slope of the curve of the sylectogram, however, it is not explained how and why the various cognitive factors are obtained.

In U.S. Pat. No. 5,003,488 an apparatus for measuring the sedimentation rate of a fluid in a sample tube is described. The apparatus comprises: a controllable element of the device of the sample and data processing means operatively coupled with the element of the device to receive sedimentation measurements produced by the element of the sedimentation device, in order to generate data on the sedimentation rate of the fluid. The element of the device of the sample includes: light source means cooperating with means to detect the light passing through the fluid, which periodically generate sedimentation measurements to determine a sedimentation rate of the fluid. Also included are means to support the test tubes, disposed between the light source means and the light detection means in order to position the test tube in which the fluid is contained.

U.S. Pat. No. 5,827,746 provides a method to determine the sedimentation rate of the corpuscles present in a blood sample; the method is carried out on a sample consisting of blood to which any suitably homogenized anti-coagulant substance has been added. The method comprises the steps of measuring an optical density, or absorbance, of a homogenized sample of blood to which an anti-coagulant has been added. The measurement takes place after rotation of the sample and as a function of time and without waiting for the formation of a plasma/corpuscle interface, in order to obtain a measurement of the optical density. The measuring step is conducted using a reading container which defines a containing chamber which contains a micro-volume of the blood sample. The internal diameter of the containing chamber guarantees a linear relationship between the optical density and the number of corpuscles present in the blood sample, in which the reading container is subjected to centrifugation during the measuring step. The measurement of the optical density is processed to obtain a sedimentation rate of the corpuscles in the blood sample. All these previous experiences, however, have not led to certain and certifiable results and, above all, have not led to obtaining a range of cognitive factors in a very short time.

By using quantitative capillary photometry, applied to blood, the present invention achieves the same result as the classical methods, and allows to obtain other cognitive factors as well, in a minimal time, around a few seconds; moreover, contrary to the state of the art, it allows to use both blood in itself and also blood rendered incoagulable.

Applicant found that, under normal conditions, the ability of red blood cells to aggregate is relatively low because repulsion forces exist between them which keep them suspended in the plasma. If the presence of globulins, or fibrinogen, or other components, is very high in the blood, the rouleaux are formed more easily and, consequently, the value of the ESR increases.

It is important to underline that the aggregation of red blood cells is a reversible phenomenon and, with adequate mixing, obtained by inverting the capped test tubes and bringing them back to their feet, with a cadence of about one second and for a number of times comprised between 12 (in the Westergreen method) and from 140 to 300, perfect de-aggregation is obtained.

This allows the measurement to be repeated for a defined number of times, at least 10 times, with negligible and independent variations on the ESR results for each blood sample.

Macromolecules, better defined as agglomerins, are present in the blood of patients suffering from a generic state of infection and reduce the forces of repulsion that move red blood cells away from each other; they also reduce the volume ratio between red blood cells and plasma, as well as increasing plasma viscosity.

Measuring ESR has three main purposes:
to report the presence of an inflammatory process,
to monitor the course or state of activity of an illness,
to identify occult pathologies.

Although it lacks specificity and sensitivity, the test is still widely used because it is cheap and easy to perform. It is also useful as a first level test because most acute and chronic inflammatory processes and processes of neoplastic diseases are associated with an increase in ESR.

To measure the ESR, the state of the art provides that the blood is previously made incoagulable. In these conditions the sedimentation of red blood cells is determined by their ability to aggregate forming the so-called rouleaux, that is, piles of cells (comparable to piles of coins) in which the various elements are linked together not by antibodies or by chemical bonds, but simply by attraction between the surfaces of the red blood cells.

On the basis of studies and research, Applicant has studied and experimented, as well applied in a secret and classified manner, for the purposes of further verification and evaluation, a new method to analyze the behavior of bodies suspended in a liquid when said bodies are stressed by forces and, in the case of blood, has been able to detect that it is possible to obtain a plurality of data, specific to that blood, using said method.

### SUMMARY OF THE INVENTION

The present invention is set forth and characterized in the independent claim. The dependent claims describe other characteristics of the present invention or variants to the main inventive idea.

We will now describe the invention in its specific application to liquid blood of mammalian-type animal origin, more specifically particularly suitable for human blood.

According to the invention, blood made incoagulable is made to pass, in a determinate minimum quantity, in a specific duct where there is a measurement window. However, native blood can also be used, for example very useful in the case of pediatric samples.

When blood is stopped in its flow, by using a peristaltic pump or another device suitable for the purpose, the red blood cells, which as they moved in the duct had assumed an elongated shape, resume their discoidal shape.

As they resume their discoidal shape, they regain the aggregation capacity determined by the possible presence of agglomerins, and such aggregation capacity varies in relation to the state of health of the carrier of that blood.

Over time, the aggregation capacity determines a curve, which although known has been studied and evaluated in different terms compared to previous uses in order to obtain other factors from the blood, which, according to the methods described in the invention, allow to obtain a new and previously not hypothesized series of data and cognitive factors from the blood, which are linked to the state of the specific individual.

In a known manner, a photometric signal is detected through the measurement window, from the moment previously defined, and the data that said signal transmits are processed for a determinate period of time.

It is also known that with the data thus obtained it is possible to generate a specific sylectogram of that blood, be it geometric or cognitive; however, with the invention we have discovered that such sylectogram allows to obtain, in an unknown manner and with a specific processing, an extensive plurality of data relating to the specific blood.

In fact, by analyzing the sylectogram generated in the predetermined unit of time, we have discovered that, by evaluating the moments of formation and the connected areas of evolution and temporal and non-temporal spaces, generated by the behavior of the aggregation of the red blood cells, it is possible to obtain the desired data by processing the factors of said areas and their generators on the basis of the method of the invention. The evaluation can be carried out qualitatively or geometrically.

From the shape of the sylectogram and from the generating factors thereof, which are connected to the measurement of said evolution, we have discovered that, if considered and processed in an appropriate manner, these factors have a direct correlation, with an insignificant margin of error, with the ESR index and with other factors of the specific blood identified below.

Applicant has, in fact, found that by measuring the time interval between the moment when the blood is stopped, in front of the measurement window (hereafter FdM), and the moment when the measurement of the ESR begins, it is possible to define the value of the elasticity of the red blood cells (Btp).

This value is characteristic of the area generated by the erythrocyte sedimentation curve.

However, according to the invention, this value can be transformed by means of a specific and previously unknown algorithm, so as to correlate this area with the erythrocyte sedimentation index sought.

Applicant was therefore able to understand, after several thousand tests, that the integration of the photometric signal, which is assumed in relation to a predefined duration, allows to obtain the ESR value and other specific values.

After studies and experiments, Applicant was also able to ascertain that the behavior of the red blood cells can be directly related, with appropriate coefficients applicable on each occasion, between the moment when the blood flow is stopped and the moment when the photometric measurement takes place.

Applicant then took into consideration the amount of time between the moment when the blood flow is stopped and the moment when it stops, and after studies and research has been able to ascertain that this amount of time can be directly related to the density of the red blood cells (Ds) by using a specific algorithm.

During the previously described studies and research, Applicant was able to define an appropriate formula which, using factors derived from the sylectogram, allows to define, by means of a special algorithm, the hematocrit value (Hct).

During the studies and research identified above, Applicant was also able to ascertain that, with a special algorithm that examines particular aspects of the sylectogram, it is possible to define the anemia factor (AnF).

Applicant, from long and extensive research and experiments on the sylectogram, as well as from the analysis of the thousands of data obtained therefrom, considering a certain area of the erythrocyte sedimentation curve, has found a specific algorithm which allows to define the erythrocyte aggregation factor (AgF).

Always based on correlated studies and research, Applicant was able to verify that, considering the behavior of the blood once stopped, it is possible to trace the viscosity (Vs) of said blood.

These three parameters, AnF, AgF and Vs, allow to confirm the real clinical state of the patient. By way of example only, an anemic patient, therefore with a low quantity of red blood cells, has in percentage fewer red blood cells that can give an ESR value correlated to the percentage of red blood cells compared to a non-anemic patient. Therefore, the measurement of the ESR, according to the invention, determined by the real presence of agglomerins, measures the real capacity of the red blood cells to aggregate, and consequently shows the true state of the sedimented red blood cells even in the presence of blood samples with few red blood cells. Therefore, Applicant's method detects the true state or behavior of the red blood cells involved in the measurement of the ESR. In summary, falsely low ESRs in anemic patients or patients with Mediterranean anemia (sickle shaped blood cells), revealed the real pathological state with the method described here.

### BRIEF DESCRIPTION OF THE DRAWINGS

Let us now see, with the aid of the attached drawings, an exemplification of the notable inventions and methodological improvements made by Applicant also using an example known sylectogram, and a purely illustrative diagram of the measuring and processing system.

The attached drawings represent:

FIG. 1 is a schematic plant for collecting data according to the present invention.

FIG. 2 shows a possible known sylectogram, which has been the subject of the extensive research.

To facilitate comprehension, the same reference numbers have been used, where possible, to identify identical common elements in the drawings, or elements fulfilling identical functions. It is understood that elements and characteristics of one embodiment can conveniently be incorporated into other embodiments without further clarifications.

### DETAILED DESCRIPTION OF SOME EMBODIMENTS

We will now refer in detail to a possible practical application of the method for embodying the invention, making use of the attached drawings which are provided by way of example only in order to better understand the invention.

Each example is supplied by way of illustration of a possible application of the invention and shall not be understood as a limitation thereof. It is understood that the present invention shall include all modifications and variants which are normal in cases of this type.

For the purposes of the invention, the attached drawings and the corresponding description supplied by way of example, can be read as follows.

By way of example, a possible rotating or fixed distributor 10 of blood test tubes 11, also and possibly containing anticoagulant, is assisted, on each occasion, by a removal mean 12, for example specific needles for each test tube 11.

These needles 12, on each occasion, are placed after washing in connection with a test tube 11 and with an analysis station 13.

This analysis station 13, by way of example, comprises a diverter 14, a pump 15, a controlled delivery system, or metering device, 17 which cooperates with a duct 18, advantageously water-repellent, which materializes at a certain point in a measurement window 19.

The measurement window 19 cooperates with a photometer 27 consisting of a light source 20 and a receiver 21.

At least the receiver 21 can be connected to a processor 22 which, on the basis of the methods and algorithms 23 contained therein, processes the data received from the photometer and supplies the results 29.

The processor controls and manages the photometer 27 and the characteristics of the light at least during the analysis.

Advantageously, the processor controls and manages the entire system.

Downstream of the measurement window 19, which with the photometer 27 constitutes the reading station 28, there can be provided a system 30 for temporarily blocking the duct 18, before the outlet 25 which flows into a waste container 26.

Advantageously, at least in correspondence with the measurement window 19, the duct 18 consists of a water-repellent capillary tube, with an internal diameter ranging from 0.8 to 8.2 mm in the example case of the tests carried out; however, these diameter values are not restrictive for the purposes of both the method and also the results.

The photometer 27 is equipped with a light source 20, the radiation of which is advantageously collimated; at least the light source 20 of the photometer 27 is advantageously controlled and managed by the processor 22.

The light source 20, in relation to the condition of the blood that is in the measurement window 19, activates the receiver 21 continuously and in a derivative manner; the receiver 21 provides detailed and continuous information to the processor 22 for the entire period of time during which the blood remains in the measurement window 19.

The processor 22 is able to manage and analyze on each occasion the information coming from the receiver 21 and provide, if necessary, the specific sylectogram.

The processor 22, according to one variant, is also able to manage the light source 20 so that at least the brightness that affects the measurement window 19, during the measurement, remains coherent with the measurement needs.

We have been able to ascertain that the same results are obtained using both native blood and also blood with anti-coagulant.

The test tubes 11, if they contain blood and anticoagulant, have to be previously and thoroughly mixed, before being placed in the distributor 10.

The pump 15, by removing, in the case of the tests, the water from the container 16, has in the meantime provided to wash the circuit upstream of the diverter 14, before the test tube 11 is placed in cooperation with the respective needle 12.

As a variant, the pump 15 can send to the metering device 17 a desired quantity of water which cleans the duct 18.

This water is also used for washing the instrument at the end of the work, while the carryover is overcome by the head-tail reading.

Such head-tail of the blood present in the duct 18 advantageously, although not necessarily, consists of air bubbles W which create the desired detachment between one blood bubble S and the other. According to one variant, a water bubble is inserted between one fraction of blood and the other.

According to the invention, a desired minimum quantity of blood is extracted from the test tube 11 which, according to the specific conditions, can have a volume from 1 to 200 μL.

For example, this desired volume can be determined by using a suction-pressure pump driven by a step motor controlled by the processor 22, or by other known mean suitable to define a predetermined or predeterminable quantity.

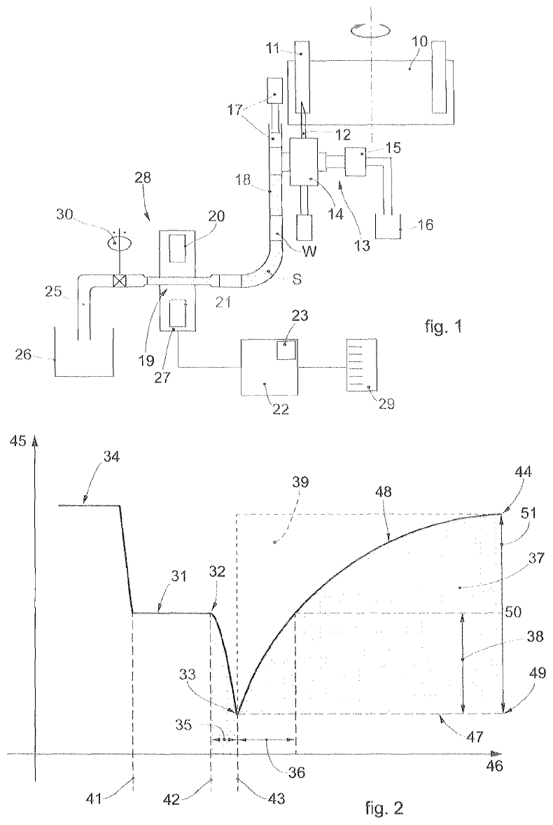

The quantity of blood S interspersed with a quantity of air or water W, which provides to clearly divide the blood samples from one another, is sent, in a cadenced and sequential manner, into the duct 18 (FIG. 1).

By way of example, FIG. 1 shows dashed segments and non-dashed segments in the duct 18. The dashed segments represent the quantity of blood S to be analyzed.

Each quantity S can be of the same blood, or of blood coming from, for example, different humans, or sources.

The clear segments W are air or water, in order to obtain the clear separation between the various blood samples.

The dashed segments represent the blood S and are of a suitable length so that the initial part of the following segment (air W) completely removes the residues of the previous segment (blood S) from the measurement window 19. In this way, it is not necessary to wash the internal part of the duct because the carryover between the samples is reduced to negligible values.

When the blood arrives in correspondence with the measurement window 19 it is stopped for the desired amount of time and the reading station 28 carries out, continuously or with a desired cadence, the reading of the luminosity that passes through the measurement window 19 over time.

The pump 15, or the mean which sends the desired quantity of blood, stops when the head of the blood to be measured is in a defined position in relation to the measurement window 19.

Therefore, the chain segment of blood S—segment of air/water W—segment of blood S—etc. advances stepwise within the desired timeframe.

The liquid to be analyzed, in the example case blood to be analyzed, is stopped in the desired position almost instantaneously and is kept still for a desired period which varies from seven to twenty seconds, advantageously from nine to twelve seconds. As soon as the allotted time is up, the blood is expelled.

To be certain that the blood stops in the desired position, it is possible to apply rapid clamping means 30 downstream of the measurement window 19.

In order to be certain that the apparatus, used for the analysis of the liquid, has a constant measurement over time, that is, it is able to analyze different liquids, it is possible to use on each occasion specific liquids calibrated in relation to the composition of the liquid with suspended bodies to be analyzed, in the example case human blood. In the case of blood, for example, such calibrated liquids can contain polymers and/or latexes, in order to calibrate, and to check the calibration of, the analysis apparatus generically described above.

These calibration liquids are read by the system as if they were the specific liquid to be analyzed, in the example case blood, generating reference values, relative to the specific liquid, which are always constant and well defined.

With this system, the instrument is both calibrated and also controlled by using different compositions that have a different turbidity generated by said polymers, or other disturbing elements; using said liquids specifically dedicated to checking the efficiency of the photometric measurement sensors, or receiver 21, according to the liquid to be analyzed.

Returning to the example case, for each portion of blood S with said system it is possible to obtain all the information described in the present invention and, if desired, it is possible to generate the corresponding and specific sylectogram.

With reference to FIG. 2, which represents an example sylectogram, we have that:
the vertical line 45 represents the value of the transmittance, measured by the photometer 27 and processed by the processor 22.
line 46 indicates time, according to the invention.
line 34 represents that the measurement window 19 is not active.
line 31, in the time line from point 41, defines respectively the moment when the new quantity of blood is starting to arrive in the measurement window 19 with a constant flow and the moment when the flow is stopped 42. Line 43 indicates the moment when the blood is completely stopped.

During the segment of line 31 the red blood cells take on an elongated shape, since they travel along the duct 18.

Point 32 is the moment when the blood is in the measurement window 19 and when the advance is blocked.

Point 32, in the predefined time corresponding to line 42, also represents the beginning of the moment when the blood stops completely, and the red blood cells are redistributed and resume their disc shape.

Point 33, in the predefined time corresponding to line 43, represents the moment when the redistribution is complete and the red blood cells have resumed their natural disc shape.

In some embodiments, an interval 35 defines the amount of time required for the red blood cells to resume the disc shape, a process which begins in point 32 and ends in point 33.

The segment between 42 and 43 therefore represents the redistribution time of the red blood cells.

Line 48 represents one of the possible decay modalities of the red blood cells that aggregate creating the rouleaux, and point 49 identifies the time reached by the curve 48 at a point 44 where the examination is provided and is finished, and the air component is made to advance before the cycle restarts with another blood sample.

Point 44 represents the transmittance value at which the red blood cells have assumed a condition of aggregation determined by the presence of agglomerins in the patient's blood, which in a few seconds expresses the correlation with the sedimentation method which, previously, with the known methods mentioned, required 1 hour. Although brief, the amount of time required by the invention is sufficient in terms of measurement.

The area 37, subtended by the line 48, represents the state of the blood the moment when the redistribution of the red blood cells in the form of rouleaux occurred in the predefined time 47.

Point 50 identifies half of an amplitude 51, while 38 represents a median index of the aggregation curve 47.

The interval 36 is the time corresponding to half of the amplitude 51.

The area 39 represents the space left free by the area 37 below the curve 48 in the rectangle defined by time 47 and amplitude 51.

By analyzing and studying this considerable amount of factors, Applicant was able to understand that:
a. the area 37 represents with great precision the sedimentation rate of the red blood cells (ESR), therefore from said area 37 the ESR can be obtained by means of an integral that calculates said area 37 knowing the time 47, the transmittance curve 48 and the value at point 44 reached at moment 49;
said area 37 is then raised to power by a factor comprised between 1.9 and 2.5 and a compensation value from 2.7 to 3.4 is subtracted from the result obtained, all being raised to a power comprised between 1.8 and 2.1;
b. the viscosity (Vs) can be obtained by subtracting the time measured at line 42 from the time measured at line 43, and multiplying the result by a reading factor comprised between 950 and 1050;
c. the elasticity of the red blood cells (Btp) can be obtained by multiplying the time of the interval 35 by a factor comprised between 120 and 130;
d. the density of the red blood cells (Ds) can be obtained by using estimation factors, using the value of the hematocrit value (Hct) multiplied by a corrective factor between 0.60 and 0.9 to which an integrative value comprised between 1000 and 1030 is added;
e. the hematocrit (Hct) can be obtained by knowing the time measured at line 43 raised to a power comprised between 3 and 4, the result of which is multiplied by a factor between 54.0 and 59.0;
f. the hemoglobin (Hb) can be obtained on the basis of the time measured at line 43 with an algorithm that provides to raise to power said time using a factor comprised between 2.2 and 2.4, multiplying the result by a factor comprised between 18.4 and 18.9;
g. the anemia factor (AnF) can be obtained again by using the time measured at line 43 minus the decay time of the calibration liquid. Said result is raised to power by a factor comprised between 4.7 and 5. The result obtained is multiplied by a factor comprised between 8.8 and 9.3. The result thus obtained is then multiplied by a factor comprised between 2.5 and 3.6;
h. the aggregation factor (AgF) is obtained by multiplying the integral of the area 37 by a factor comprised between 0.001 and 0.003.

It is clear that modifications may be made to the method as described heretofore, without departing from the field and scope of the present invention.

It is also clear that, although the present invention has been described with reference to blood using some specific examples, a person of skill in the art shall certainly be able to achieve many other equivalent applications of system and method with respect to what has been described here. Such applications being able to be dedicated to other specific liquids containing suspended bodies subjected to forces.

In the following claims, the sole purpose of the references in brackets is to facilitate reading: they must not be considered as restrictive factors with regard to the field of protection claimed in the specific claims.

1. A method for the analysis of a liquid with suspended bodies, stressed by forces, said method using at least one test-tube container element containing whole or conditioned blood, said container being advantageously present in a distributor, a mean for removing and delivering a desired quantity of said blood in a controlled manner, said desired quantity being preceded and followed by a separator bubble, said duct and said bubbles not interacting with the blood, said duct being connected to a measurement window where said duct is calibrated, said measurement window being associated with a photometer cooperating with a processor for processing the information detected by the photometer, said blood remaining in the measurement window for a predetermined and controlled period of time, the processor generating on each occasion a connected sylectogram containing parametric information supplied by the photometer which is processed in geometric form and/or analytical form, said parametric information connected to the specific blood is processed on the basis of specific algorithms configured to obtain one or more of the following factors: Viscosity; Elasticity of the suspended bodies; Density of the liquid; Hematocrit value; Hemoglobin; Anemia factor and Aggregation factor.

2. The method for the analysis of a specific blood as in claim 1, wherein the ESR is obtained by measuring an area subtended by part of the sylectogram and raising to power said area by a factor comprised between 1.9 and 2.5, a compensation value between 2.7 and 3.4 being subtracted from the result obtained, all being raised to a power comprised between 1.8 and 2.1.

3. The method for the analysis of a liquid as in claim 1, wherein the Viscosity is obtained by subtracting from the time measured in correspondence with a line of formation of the sylectogram, the time measured in correspondence with a line of the same sylectogram, and multiplying the result by a factor comprised between 950 and 1050.

4. The method for the analysis of a liquid as in claim 1, wherein the elasticity of the red blood cells is obtained by multiplying the interval between the positions and of the sylectogram by a factor comprised between 120 and 130.

5. The method for the analysis of a liquid as in claim 1, wherein the hematocrit is obtained by using the time measured in correspondence with the line of the sylectogram raised to a power comprised between 3 and 4, the result of which is multiplied by a factor between 54 and 59.0.

6. The method for the analysis of a liquid as in claim 1, wherein the density of the red blood cells is obtained by using the value multiplied by a corrective factor between 0.6 and 0.9 to which an integrative value comprised between 1000 and 1030 is added.

7. The method for the analysis of a liquid as in claim 1, wherein the hemoglobin is obtained on the basis of the time measured at the line of the sylectogram raising it to power by a value comprised between 2.2 and 2.4, and multiplying the result by a factor comprised between 18.4 and 18.9.

8. The method for the analysis of a liquid as in claim 1, wherein the anemia factor is obtained by using the time measured in correspondence with the line of the sylectogram minus the decay time of the calibration liquid, said result being raised to power by a factor comprised between 4.7 and 5, the result is then multiplied by a value comprised between 8.8 and 9.3 and the result obtained is multiplied by a factor comprised between 2.5 and 3.6.

9. The method for the analysis of a liquid as in claim 1, wherein the aggregation factor is obtained by multiplying the integral of the area of the sylectogram by a factor comprised between 0.001 and 0.003.
